(19) United States
(12) Patent Application Publication
(10) Pub. No.: US 2014/0336716 A1
(43) Pub. Date: Nov. 13, 2014

(54) MEDICAL SPRING DEPLOYMENT DEVICE AND INSERTION TOOL

(71) Applicant: OsteoMed LLC, Addison, TX (US)

(72) Inventors: Chuck Seegert, Carrollton, TX (US); Jason C. Kilmer, Richardson, TX (US)

(73) Assignee: OsteoMed LLC, Addison, TX (US)

(21) Appl. No.: 13/889,081

(22) Filed: May 7, 2013

(51) Int. Cl. A61B 17/88

(52) U.S. Cl. CPC A61B 17/8872; A61B 2017/681
USPC 606/86 R; 29/434

(57) ABSTRACT

Systems, methods and devices which may be utilized in surgical treatments that insert a spring-force based separating device configured to maintain separation of two bone portions are provided. In one embodiment, a security device may be provided which is configured to house a spring in a compressed state for insertion into a patient. Upon insertion of the spring, the security device may be configured to release the compression force on the spring to allow the spring provide outward force on bone surfaces of a patient. Additionally, the security device may be configured to release the spring after the spring is deployed in the patient and to allow for removal of the security device. Embodiments may also include an insertion tool configured to utilize a security device to deploy a spring.

### TECHNICAL FIELD

The present application relates to systems, methods and devices for maintaining separation of bone structures, such as for treating craniosynostosis.

### BACKGROUND

Craniosynostosis is a condition in which one or more of the fibrous sutures in an infant skull prematurely fuses by turning into bone (ossification), thereby changing the growth pattern of the skull. When such a condition occurs, because the skull cannot expand perpendicular to the fused suture, it may compensate by growing more in the direction parallel to the closed sutures. Sometimes the resulting growth pattern provides the necessary space for the growing brain, but it may result in an abnormal head shape and abnormal facial features. In cases in which the compensation does not effectively provide enough space for the growing brain, craniosynostosis may result in increased intracranial pressure leading possibly to visual impairment, sleeping impairment, eating difficulties, or an impairment of mental development combined with a significant reduction in IQ.

Many methods for treating craniosynostosis have been implemented. One relatively new method that is gaining acceptance entails creating a strip craniectomy and inserting a device configured to maintain separation of the newly-defined areas of the skull. In such a procedure, a portion of the skull (e.g. a 1 cm wide section) is removed and spring is placed into the void generated by the removal of the skull portion, which then exerts outward force on the skull from within the removed portion in order to facilitate the proper, more anatomical, growth vector in the skull.

While such a treatment has shown to be a marked improvement over earlier methods, some issues may still arise which are associated with this procedure. For example, a strip craniectomy may be invasive to the patient because of the space required (and corresponding length of incision) to remove the bone, etc. Generally incision length of this size is required because the surgical instruments being employed are general designs and not specifically suited for this procedure. To get proper access using these instruments, larger incisions are made. Further, the placement of the spring may be difficult due to the space needed to compress the spring and to insert the spring into the removed bone area. In fact, in some cases, the placement of the spring may even require multiple surgeons.

It is also appreciated that there are a number of additional problems which are associated with higher invasiveness of a surgery. For example, additional complexity of a surgery may result in longer anesthesia times for the patient undergoing the surgery. Increased anesthesia doses and time may increase possible adverse effects resulting from the anesthesia drugs. Further, longer surgery times and larger incisions may cause additional blood loss to occur, as well as, increase the time required for patient healing.

### BRIEF SUMMARY

The present application provides for systems, methods and devices which may be utilized in surgical treatments that insert a spring-force based separating device configured to maintain separation of two bone portions. In one embodiment, a security device may be provided which is configured to retain a spring in a compressed state for insertion into a patient. Upon insertion of the spring, the security device may be configured to release the compression force on the spring to allow the spring provide outward force on bone surfaces of a patient. Additionally, the security device may be configured to release the spring after the spring is deployed in the patient and to allow for removal of the security device.

Embodiments may allow for a spring to be deployed from a single incision which is perpendicular with respect to an axis defined by the direction of insertion of the spring. Further, embodiments may allow for a plurality of springs to be deployed from the single incision which is perpendicular with respect to an axis defined by the direction of insertion of the spring, e.g. to allow a spring to be inserted in both perpendicular directions.

In another embodiment an insertion tool may be provided which attaches to a security device and assists in the insertion of a spring device. The insertion tool may comprise a sliding and/or trigger mechanism which allows a compressed spring to extend out of the end of the security device and to become uncompressed. Such a mechanism may be implemented as a slidably coupled railing system whereupon the inserter may cause the railing system to contract away from the end of the security device while a second portion of the railing system causes the spring to extend from the end of the security device.

In accordance with one embodiment a system is provided for deploying a spring device into a patient. The system includes a security device having a retaining portion and a sleeve portion. The retaining portion is configured to retain the spring device prior to deployment and is further configured to be slidably disposed within said sleeve portion. Additionally, the sleeve portion is configured to provide compression force on the retaining portion and spring device when the retaining portion is disposed within the sleeve portion. The system further includes an inserter configured to attach to the security device and to slidably retract the sleeve portion of the security device. The inserter further includes an extension portion configured to provide force on the retaining portion to prevent the retaining portion from retracting with the sleeve portion, thereby causing the retaining portion to extend out of the sleeve portion when the sleeve portion is retracted.

In accordance with another embodiment, a device for deploying a spring force between two bone structures in a patient is provided. The device includes a retaining portion configured to retain a spring device for deployment into a patient. The device further includes a sleeve portion configured to slidably accept the retaining portion, wherein the sleeve portion is configured to provide compression force on the retaining portion and the spring device when the retaining portion is disposed within the sleeve portion. Additionally, the retaining portion is configured to slidably extend from the end of the sleeve portion to release the compression force of the sleeve portion on the spring device.

In another embodiment a device for deploying a spring force between two bone structures in a patient is provided. This device includes an inserter tool configured to attach to a security device. The inserter tool includes a sliding portion and an extension portion. The sliding portion is configured to transfer a pulling force to a first portion of an attached security device, while the extension portion remains unmoved and provides a stopping force to second portion of the attached security device to prevent pulling on the second portion.

Another embodiment provides for a method for forming a spring deployment system. The method includes forming a retaining portion configured to retain a spring device for deployment into a patient. Additionally, the method includes forming a sleeve portion configured to slidably accept the retaining portion. The sleeve portion may be configured to provide compression force on the retaining portion and the spring device when the retaining portion is disposed within the sleeve portion. The retaining portion is also configured to slidably extend from the end of the sleeve portion to release the compression force of the sleeve portion on the spring device. In accordance with a more detailed embodiment, the method may also include forming an inserter tool configured to attach to the sleeve portion which is configured to slidably retract the sleeve portion. The inserter is further formed with an extension portion configured to provide force on said retaining portion to prevent the retaining portion from retracting with the sleeve portion, thereby causing the retaining portion to extend out of the sleeve portion when the sleeve portion is retracted.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

For a more complete understanding, reference is now made to the following descriptions taken in conjunction with the accompanying drawings, in which:

FIG. 1 illustrates a spring device insertion system in accordance with an embodiment of the present application;

FIG. 2 illustrates a security device in accordance with an embodiment of the present application;

FIG. 3 illustrates an assembled security device in accordance with an embodiment of the present application;

FIG. 4 illustrates a spring insertion device in accordance with an embodiment of the present application;

FIG. 5 illustrates an exploded view or the spring insertion device of FIG. 4;

FIG. 6 illustrates spring device insertion system in accordance with an embodiment of the present application;

FIG. 7 illustrates spring device insertion system in accordance with an embodiment of the present application; and

FIG. 8 illustrates spring device insertion system in accordance with an embodiment of the present application.

### DETAILED DESCRIPTION

As an initial matter it is noted that much of the following discussion is made in the context of inserting a spring into a gap in the skull which has been made in the course of a craniectomy. Such discussion is made for the sake of clearly describing the concepts herein. It is noted that the inventive methods may be utilized in other portions of the body where separation between two structures by means of a spring force is implemented. It is further noted that the term “spring” is utilized throughout the present application, and that a spring may entail any device which may function to be compressed in order to facilitate insertion and act to provide an outward force (or spring force) on opposing surfaces when deployed.

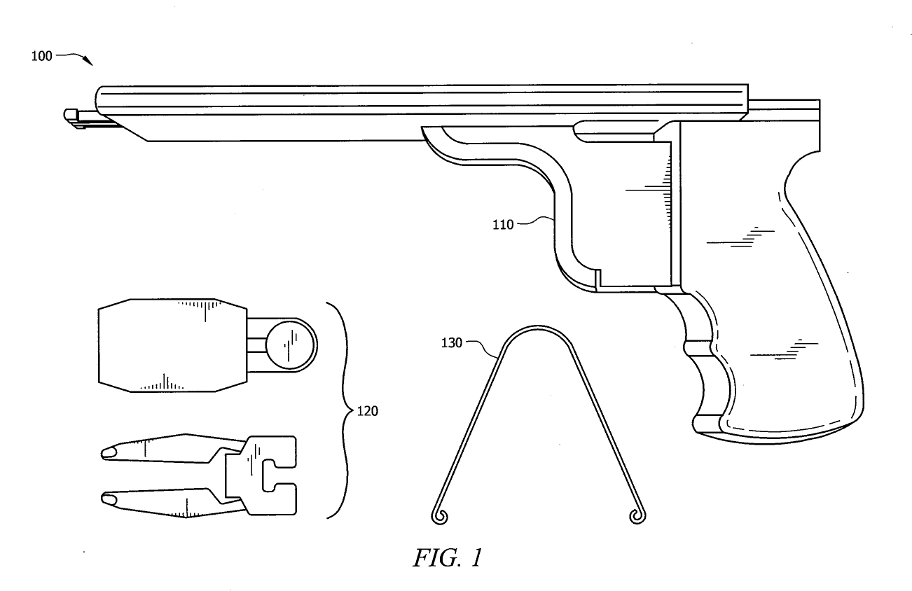

FIG. 1 illustrates a spring device insertion system 100 in accordance with an embodiment of the present invention. Spring device insertion system 100 includes an inserter 110, a security device 120 and a flexible spring 130. In some embodiments, spring device insertion system 100 may include a plurality of one or more of components 110-130. For example, in some embodiments multiple flexible springs 103 may be provided for insertion into a patient. In such circumstances, a second spring security device 120 may be utilized for an additional spring 130. Alternatively, one or more of the devices of system 100 may be configured to be reusable during a single surgery when additional devices are utilized, e.g. inserter 110 may be reused when inserting a second flexible spring 130 within retained within a second security device 120. System 100 may be provided in this manner as a kit for use by a surgeon in the course of treating a condition such as craniosynostosis.

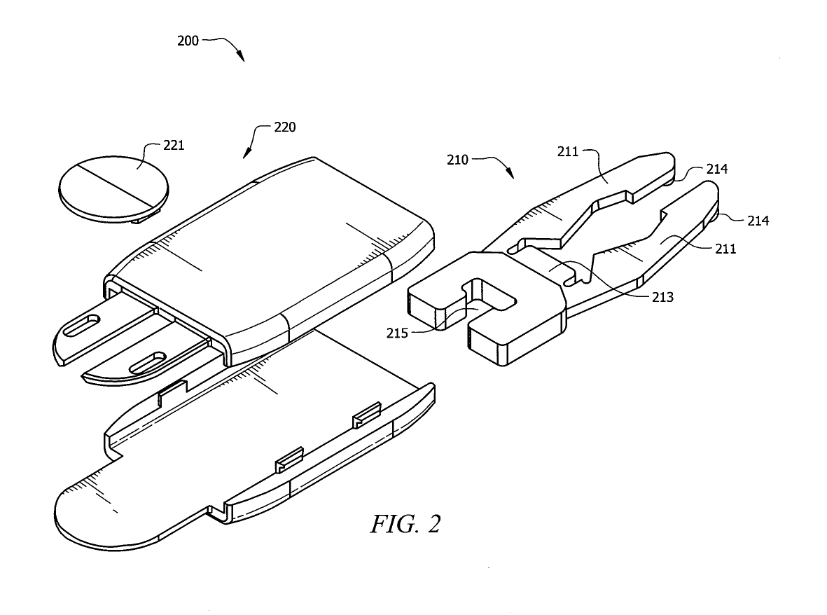

FIG. 2 illustrates a security device 200 in accordance with an embodiment of the present application. Security device 200 includes a retainer portion 210 and a sleeve portion 220. Retainer portion 210 is configured to slidably engage with sleeve portion 220.

In the illustrated embodiment, retainer portion 210 comprises first and second arms 211 and a release area 213. First and second arms 211 may include one or more guide surfaces 214 configured to retain a spring (not shown). Additionally, first and second arms 211 may be configured to be flexible to allow for compression and decompression of a spring which is secured within device 200. For example, one or more of first and second arms 211 may be configured to pivotably flex at an angle away from axis X in order to facilitate such compression/decompression of a spring device.

Release area 213 may be configured to retain a spring while the release area 213 of retainer portion 210 is contained within sleeve portion 220. When retainer portion 210 is allowed to slide such that release area 213 of retainer portion 210 extends out of sleeve portion 220, the spring may be disengaged from security device 200 for deployment into a patient.

Retainer portion 210 of security device 200 further includes a cavity 215 configured to accept an extension from an insertion device. Such an extension may be configured to provide force to allow retainer portion 210 to slide within sleeve portion 220. Accordingly, cavity 215 may be configured to accept the extension in a snap fit configuration or in any other configuration which will allow the extension to transfer force to retainer portion 210 travelling within sleeve portion 220 such as, for example, a force sufficient to slide retainer portion into and/or out of sleeve portion 220 when a compressible spring is retained within retainer portion 210.

Sleeve portion 220 of security device 200 is configured to contain retainer portion 210 and configured to provide compression forces for a spring disposed within retainer portion 210. For example, as retainer portion 210 (including a spring) is drawn into sleeve portion 220, the outer walls of sleeve portion 220 provide force to compress at least a portion of the spring toward the X axis. Conversely, as retainer portion 210 is extended outside of sleeve portion 220, the walls of sleeve portion 220 no longer provide force to compress the spring and the spring is allowed to extend outward with respect to the X axis.

Sleeve portion 220 is further configured to connect to an inserter device such that sleeve portion 220 may remain affixed to the inserter device while an extension received in cavity 215 of retainer portion 210 may be utilized to slidably displace retainer portion 210 within sleeve portion 220 of security device 200. This connection may be secured using a button device 221 which is configured to engage with at least one surface on the inserter device. Additionally, the connection of sleeve portion 220 to an inserter device may be implemented so as to allow the connection of security device 200 to an inserter to be a releasable connection. It is noted that while a button connection is illustrated, any connection which will reasonably retain the security device while allowing for an inserter to transmit force to retainer portion 210 may be utilized.

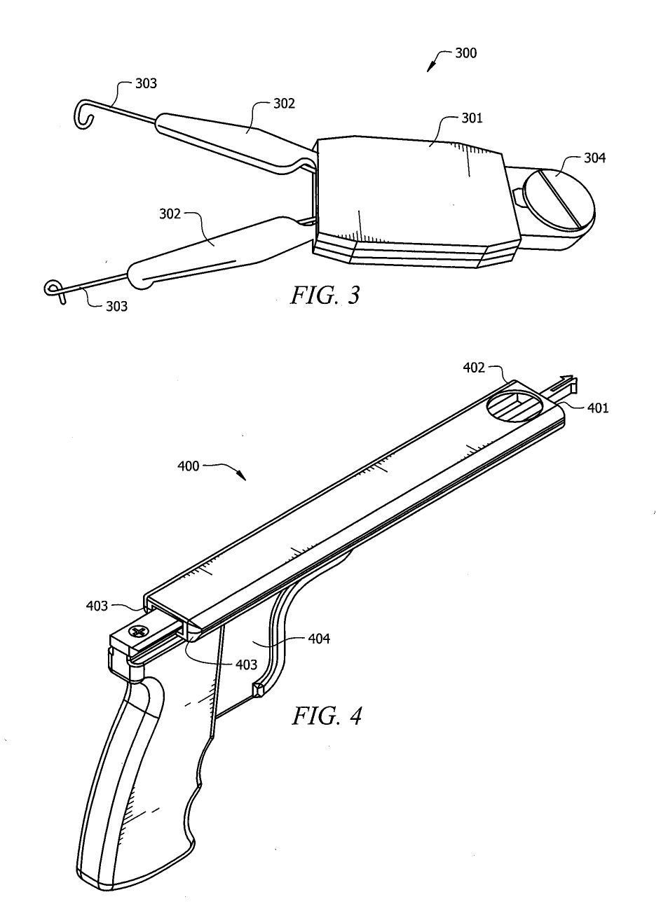

FIG. 3 illustrates an assembled security device 300 in accordance with an embodiment of the present invention. Security device includes a sleeve portion 301, a spring retainer portion 302, spring 303 and inserter connection portion 304. Retainer portion 302 is configured to slide with respect to sleeve portion 301. As can be seen, in the event that retainer portion 302 were to slide further within sleeve portion 301, the arms of spring 303 (and retainer portion 302) would be drawn in together to be contained within sleeve portion 301. Such a compression allows for a compact insertion of spring 303 which is advantageous for multiple reasons, e.g. allows the security device and spring to be inserted while not requiring a large incision into a patient, allows for easier access for a surgeon to deploy the spring in an unassisted manner, etc. Security device 300 is configured to affix sleeve portion 301 to an inserter device via inserter connection portion 304 while also allowing the inserter to provide force to retainer portion 302 in order to slidably insert or remove the security device 300 (and to insert spring 303) from the patient.

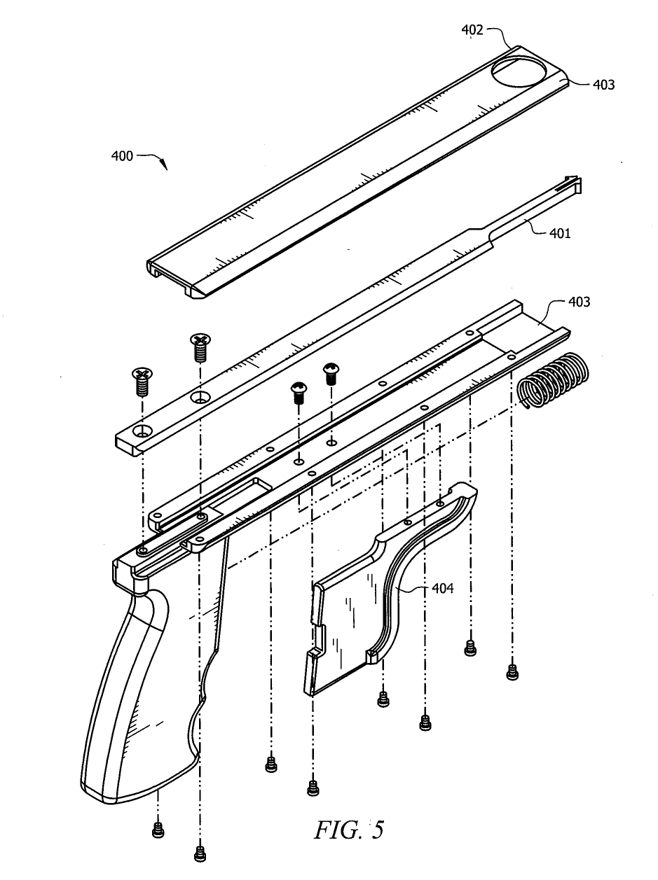

FIG. 4 illustrates a spring insertion device 400 in accordance with an embodiment of the present application. FIG. 5 illustrates an exploded view of spring insertion device 400. Spring insertion device 400 includes an extension portion 401 configured to engage with a security device (e.g. such as within cavity 215 of FIG. 2) and to facilitate slidable motion of a retainer portion of the security device.

It is noted that while extension portion is shown as having snap fit flanges, any means to connect the extension portion 401 to the security device may be utilized without departing from the concepts of the present invention. It is appreciated that such a connection would be of sufficient strength so as to maintain the connection with the security device (such as with a retainer portion) while being moved with respect to other portions of the security device (such as a sleeve portion).

Spring insertion device 400 also includes a security device connection portion 402. In the illustrated embodiment, security device connection portion 402 is an aperture configured to receive a button style device (e.g. button device 221) of a security device in order to affix the security device to spring insertion device 400 such that a sleeve portion of the security device may be pulled by spring insertion device 400 without releasing the security device. It is appreciated that any type of connection between a security device and spring insertion device 400 may be utilized which allows for this functionality. The illustrated embodiment offers advantages in terms of simplicity and the ability to be functional while maintaining a low profile.

It is further noted that this connection may be implemented in a manner that is easily releasable. For example, a button device may be configured such that a user may push on the button to release the security device. Such a configuration may provide for interchanging multiple security devices (e.g. in the event that multiple springs are to be inserted).

Spring insertion device 400 includes a sliding rail portion 403 where a portion of the inserter may slide with respect to another portion of the inserter. Such sliding may be facilitated by squeezing trigger 404 and/or by manually pulling on a portion of sliding rail portion 403. It is noted that the portion of spring insertion device 400 that is connected to a security device 402 will slide with respect to extension portion 401. Such motion allows for the extension and retraction of a retainer portion of the security device and for eventual deployment of a spring as a result of extension portion 401 remaining in a fixed position with respect to sliding portion 403 and providing a force onto a retainer portion while an opposite force is exerted on a sleeve portion of a security device.

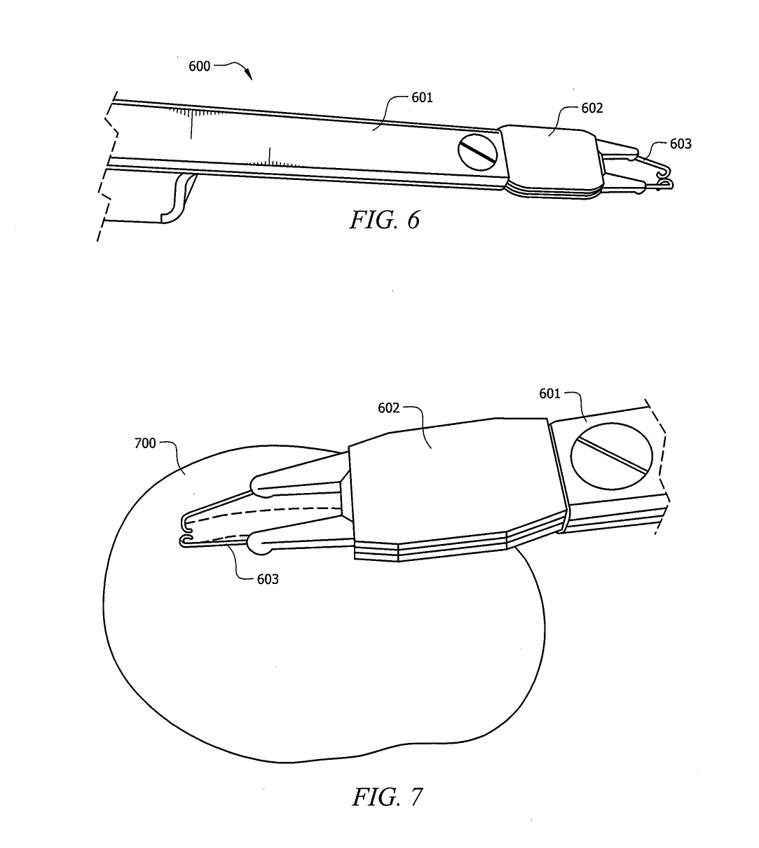

FIGS. 6-8 illustrate a system 600 in various stages of deployment in accordance with an embodiment of the present application. System 600 includes spring insertion device 601, security device 602 and spring 603. In FIG. 6, spring 603 and retaining portion of security device 602 are drawn into the sleeve portion such that spring 603 is in a compressed form. In this form, a surgeon may create an incision which allows for spring 603 and at least a portion of security device 602 to be inserted within the incision for the deployment of spring 603. This method provides a substantial improvement over previous methods where more exposure to the skull is needed to deploy spring 603, and where deployment may require multiple people to implement.

FIG. 7 illustrates system 600 in the process of an implantation of spring 603 into skull 700. Spring 603 is navigated to its correct placement using insertion device 601. Once spring 603 is in its proper place (e.g. when hooks on the end of spring 603 are in contact with the opposing sides of the skull), a user may pull the slide of insertion device 601 which causes sleeve portion of security device 602 to retract with the sliding motion of insertion device 601, thereby causing retainer portion of security device 602 to extend away from sleeve portion. Upon retainer portion of security device 602 extending away from the sleeve portion, spring 603 will undergo a reduction in compression force and attempt to motion to a relaxed state. In this manner, the hooks on the end of spring 603 which are engaged with the skull will be held in place by the spring force of spring 603.

It is appreciated that the present application discusses motion of the retainer portion in terms of the retainer portion extending and the sleeve portion retracting. It is noted that this extension and retraction is generally discussed with respect to each other and it is not intended that the movement be limiting on various placements and motions which will be utilized for insertion of a spring device. For example, the retainer portion may be placed in a desired location whereupon the sleeve portion is retracted without moving the spring portion. Further, the retainer portion may also undergo extension movement during insertion due to the nature of how the spring device may need to be employed. In any event, the retainer portion is extending/retracting with respect to the sleeve portion of a security device.

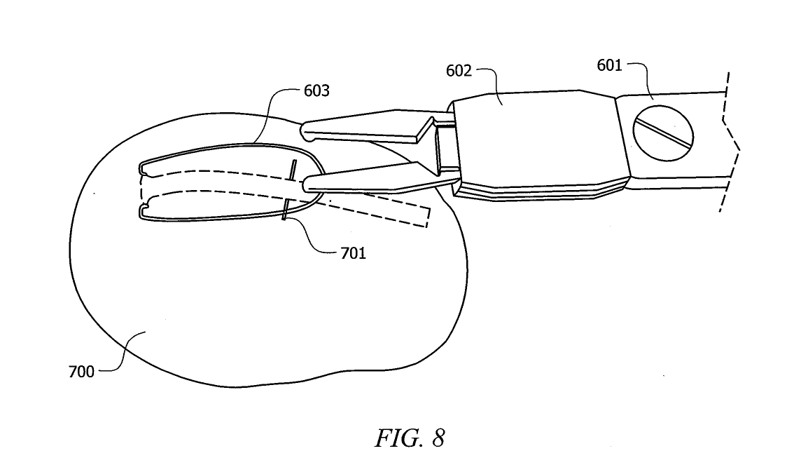

FIG. 8 illustrates system 600 after the retainer portion of security device 602 has been fully extended and spring 603 has been deployed. As can be seen, once the retainer portion of security device 602 is extended past a certain point (e.g. where release area 213 is extended outside of the sleeve portion) the spring may be disengaged from security device 602. After the spring is disengaged, security device 602 may be removed from the patient at the incision area (denoted by 701).

As discussed above, embodiments may allow for multiple springs to be inserted, and in some cases, multiple springs may be deployed from the same incision site. For example, referring to FIG. 8, a second spring may be inserted utilizing a same incision area 701 where the spring is deployed to extend toward the back side of skull 700.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A system for deploying a spring device into a patient, said system comprising:
a security device having a retaining portion and a sleeve portion, said retaining portion configured to retain the spring device prior to deployment, said retaining portion further configured to be slidably disposed within said sleeve portion, wherein said sleeve portion is configured to provide compression force on said retaining portion and spring device when said retaining portion is disposed within said sleeve portion; and
an inserter configured to attach to said security device and configured to slidably retract said sleeve portion of said security device, said inserter further including an extension portion configured to provide force on said retaining portion to prevent said retaining portion from retracting with said sleeve portion, thereby causing the retaining portion to extend out of the sleeve portion when the sleeve portion is retracted.

2. The system of claim 1 further comprising a spring device configured to seat within said retaining portion and to be compressed when retaining portion is within the sleeve portion and configured to move toward a relaxed state when slidably exiting the sleeve portion.

3. The system of claim 1 further comprising a plurality of security devices and springs configured to be utilized with a single inserter to deploy a plurality of springs in a single patient.

4. The system of claim 1 wherein said security device and inserter are configured to insert the spring device from a single perpendicular incision point.

5. The system of claim 1 wherein the inserter further comprises a trigger portion configured to be squeezed by a user to create the slidably retracting force on said sleeve portion of said security device.

6. The system of claim 1 wherein said retaining portion is configured to release the spring for deployment when said retaining portion is fully extended out of said sleeve portion.

7. A device for deploying a spring force between two bone structures in a patient, said device comprising:
a retaining portion, said retaining portion configured to retain a spring device for deployment into a patient; and
a sleeve portion, said sleeve portion configured to slidably accept said retaining portion, wherein said sleeve portion is configured to provide compression force on said retaining portion and said spring device when said retaining portion is disposed within said sleeve portion;
wherein said retaining portion is configured to slidably extend from the end of said sleeve portion to release the compression force of the sleeve portion on said spring device.

8. The device of claim 7 wherein the retaining portion further comprises a first and second arm, at least one of said first and second arms having one or more guide surfaces configured to retain said spring device.

9. The device of claim 7 wherein the retaining portion further comprises a release area configured to retain said spring device until the retaining portion is slidably extended away from said sleeve portion to a point where said spring device is disengaged from said retaining portion.

10. The device of claim 7 wherein said spring device comprises one or more hooks configured to secure the spring device between the two bone structures of the patient.

11. The device of claim 7 further comprising an attachment mechanism configured to facilitate an attachment of said device with an insertion device.

12. The device of claim 7 wherein the retaining portion defines a cavity configured to accept an extension portion of an inserter device.

13. A device for deploying a spring force between two bone structures in a patient, said device comprising:
an inserter tool configured to attach to a security device, said inserter tool including a sliding portion and an extension portion, said sliding portion configured to transfer a pulling force to a first portion of an attached security device while said extension portion remains unmoved and provides a stopping force to second portion of the attached security device to prevent pulling on the second portion.

14. The device of claim 13 wherein said inserter tool further comprises a trigger portion configured to be squeezed by a user to create the pulling force on the first portion of the attached security device.

15. The device of claim 13 wherein the inserter tool is configured to attach to a security device with an aperture configured to receive a button disposed on the security device.

16. The device of claim 15 wherein the button device is configured to be pressed out of the aperture to detach the security device.

17. The device of claim 13 wherein the extension portion is configured to snap fit with the second portion of the attached security device.

18. A method for forming a spring deployment system, said method comprising:
forming a retaining portion configured to retain a spring device for deployment into a patient; and
forming a sleeve portion, said sleeve portion configured to slidably accept said retaining portion, wherein said sleeve portion is configured to provide compression force on said retaining portion and said spring device when said retaining portion is disposed within said sleeve portion, and wherein said retaining portion is configured to slidably extend from the end of said sleeve portion to release the compression force of the sleeve portion on said spring device.

19. The method of claim 18 further comprising forming the retaining portion with a first and second arm, at least one of said first and second arms having one or more guide surfaces configured to retain said spring device.

20. The method of claim 18 further comprising forming the retaining portion with a release area configured to retain said spring device until the retaining portion is slidably extended away from said sleeve portion to a point where said spring device is disengaged from said retaining portion.

21. The method of claim 18 further comprising forming the sleeve portion with an attachment mechanism configured to facilitate an attachment of said sleeve portion with an insertion device.

22. The method of claim 18 wherein the retaining portion is formed with a cavity configured to accept an extension portion of an inserter device.

23. The method of claim 18 further comprising forming an inserter tool configured to attach to said sleeve portion and configured to slidably retract said sleeve portion, said inserter further formed with an extension portion configured to provide force on said retaining portion to prevent said retaining portion from retracting with said sleeve portion, thereby causing the retaining portion to extend out of the sleeve portion when the sleeve portion is retracted.
